(19) United States
(12) Patent Application Publication
(10) Pub. No.: US 2025/0391533 A1
(43) Pub. Date: Dec. 25, 2025

(54) ELECTRONIC DEVICE, METHOD, AND STORAGE MEDIUM FOR PROVIDING INFORMATION ON MEDICATION

(71) Applicant: SAMSUNG ELECTRONICS CO., LTD., Suwon-si (KR)

(72) Inventors: Sehee LEE, Suwon-si (KR); Eunhye LEE, Suwon-si (KR); Eunmi SHIN, Suwon-si (KR); Hyunjin SHIN, Suwon-si (KR); Cheolkyu LIM, Suwon-si (KR)

(21) Appl. No.: 19/094,072

(22) Filed: Mar. 28, 2025

Related U.S. Application Data
(63) Continuation of application No. PCT/KR2025/002530, filed on Feb. 24, 2025.

(30) Foreign Application Priority Data
Jun. 24, 2024 (KR) 10-2024-0082182
Aug. 1, 2024 (KR) 10-2024-0102550

(51) Int. Cl. G16H 20/10; G16H 40/67

(52) U.S. Cl. CPC G16H 20/10; G16H 40/67

(57) ABSTRACT

An electronic device, method, and storage medium that provide information on a medication are provided. The electronic device includes: a display, at least one processor, comprising processing circuitry, and memory storing instructions, wherein the instructions, when executed by the at least one processor individually or collectively, cause the electronic device to: receive information on a medication, based on a first input; obtain a medication category related to the medication, based on the information on the medication; receive a second input indicating information on a time at which the medication was taken; obtain biometric information of a user; based on the biometric information and the information on the time at which the medication was taken, obtain a first score according to a first factor applicable to a plurality of medication categories and a second score according to a second factor applicable to the obtained medication category related to the medication; and provide, through the display, a first visual object indicating the first score according to the first factor and the second score according to the second factor.

### CROSS-REFERENCE TO RELATED APPLICATIONS

This application is a continuation of International Application No. PCT/KR2025/002530 designating the United States, filed on Feb. 24, 2025, in the Korean Intellectual Property Receiving Office and claiming priority to Korean Patent Application Nos. 10-2024-0082182, filed on Jun. 24, 2024, and 10-2024-0102550, filed on Aug. 1, 2024, in the Korean Intellectual Property Office, the disclosures of each of which are incorporated by reference herein in their entireties.

### BACKGROUND

### Field

The disclosure relates to an electronic device, method, and storage medium that provide, for example, information on a medication.

### Description of Related Art

In addition to hand-held type electronic devices, wearable type electronic devices worn on the user's body are becoming popularized. Various types of electronic devices may include a function of measuring the user's bio-signals. As interest in health care increases, electronic devices may provide analysis information on measured bio-signals, ingested items (e.g., food, medication, etc.), and/or health status.

The above information may be presented as background information simply to assist with an understanding of the disclosure. No determination has been made, and no assertion is made, as to whether any of the above might be applicable as prior art with regard to the disclosure.

### SUMMARY

According to various example embodiments of the disclosure, an electronic device may include: a display, at least one processor including processing circuitry, and memory storing instructions, wherein the instructions, when executed by the at least one processor individually or collectively, cause the electronic device to: receive information on a medication, based on a first input; obtain a medication category related to the medication, based on the information on the medication; receive a second input indicating information on a time at which the medication was taken; obtain biometric information of a user; based on the biometric information and the information on the time at which the medication was taken, obtain a first score according to a first factor applicable to a plurality of medication categories and a second score according to a second factor applicable to the obtained medication category related to the medication; and provide, through a display, a first visual object indicating the first score according to the first factor and the second score according to the second factor.

According to various example embodiments of the disclosure, a method of providing information on a medication may include: receiving information on a medication, based on a first input; obtaining a medication category related to the medication, based on the information on the medication; receiving a second input indicating information on a time at which the medication was taken; obtaining biometric information of a user; based on the biometric information and the information on the time at which the medication was taken, obtaining a first score according to a first factor applicable to a plurality of medication categories and a second score according to a second factor applicable to the obtained medication category related to the medication; and providing, through a display, a first visual object indicating the first score according to the first factor and the second score according to the second factor.

According to various example embodiments of the disclosure, a non-transitory computer-readable storage medium having a program, recorded thereon, wherein the program, when executed by at least one processor of an electronic device, comprising processing circuitry, individually and/or collectively, cause the electronic device to perform a method for providing information on a medication, including: receiving information on a medication, based on a first input; obtaining a medication category related to the medication, based on the information on the medication; receiving a second input indicating information on a time at which the medication was taken; obtaining biometric information of a user; based on the biometric information and the information on the time at which the medication was taken, obtaining a first score according to a first factor applicable to a plurality of medication categories and a second score according to a second factor applicable to the obtained medication category related to the medication; and providing, through a display, a first visual object indicating the first score according to the first factor and the second score according to the second factor.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a block diagram illustrating an example electronic device in a network environment according to various embodiments;

FIG. 2 is a block diagram illustrating an example configuration of an electronic device according to various embodiments;

FIGS. 3A, 3B, 3C, 3D, 3E, and 3F are diagrams illustrating an example operation of adding information on a taken medication according to various embodiments;

FIGS. 4A and 4B are diagrams illustrating an example operation of managing a schedule for taking a medication according to various embodiments;

FIG. 5 is a diagram illustrating an example system for receiving biometric information from a wearable device according to various embodiments;

FIGS. 6A, 6B, 6C, 6D, and 6E are diagrams illustrating example UIs of analysis data according to various embodiments;

FIG. 7 is a diagram illustrating example UIs before and after an effect onset time of a taken medication according to various embodiments;

FIGS. 8A and 8B are diagrams illustrating an example operation of receiving information from a user according to various embodiments;

FIG. 9 is a diagram illustrating an example UI of analysis data including side effect information according to various embodiments;

FIGS. 10A and 10B are diagrams illustrating an example operation of providing information on related medications according to various embodiments;

FIG. 11 is a diagram illustrating an example dosage time reminder UI after receiving side effect information according to various embodiments;

FIG. 12 is a diagram illustrating an example operation of providing comparative data before and after taking a medication according to various embodiments; and

FIG. 13 is a flowchart illustrating an example method for providing information on a taken medication according to various embodiments.

### DETAILED DESCRIPTION

Hereinafter, various example embodiments of the disclosure will be described in greater detail with reference to the drawings. However, the disclosure may be implemented in various different forms and is not limited to the example embodiments described herein. In relation to the description of the drawings, the same or similar reference numerals may be used for the same or similar components. In addition, in the drawings and related descriptions, descriptions of well-known functions and configurations may be omitted for clarity and conciseness.

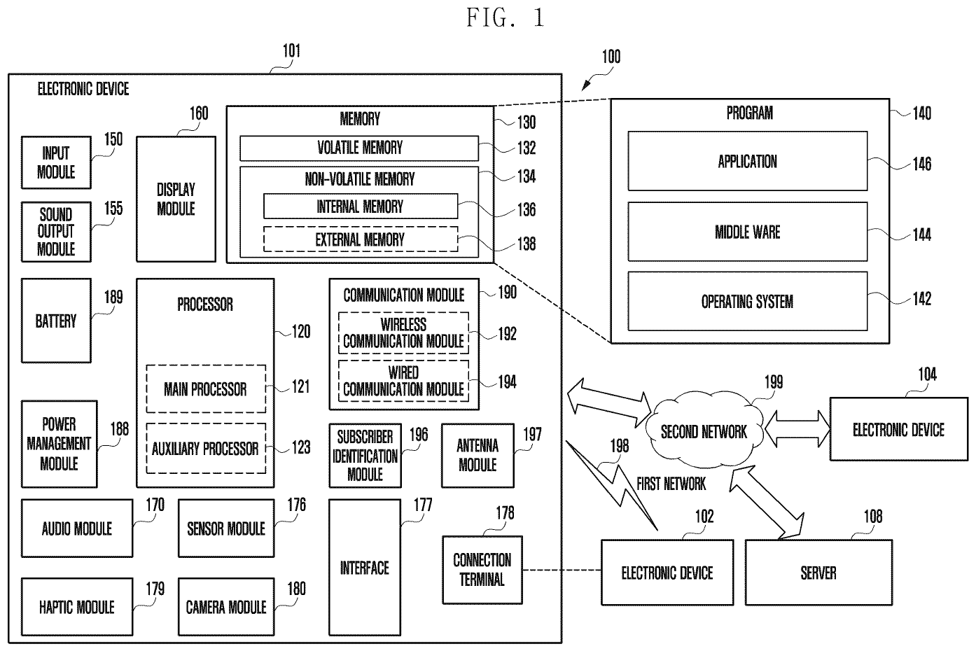

FIG. 1 is a block diagram illustrating an example electronic device 101 in a network environment 100 according to various examples. Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an example, the electronic device 101 may communicate with the electronic device 104 via the server 108. According to an example, the electronic device 101 may include a processor 120, memory 130, an input module 150, a sound output module 155, a display module 160, an audio module 170, a sensor module 176, an interface 177, a connection terminal 178, a haptic module 179, a camera module 180, a power management module 188, a battery 189, a communication module 190, a subscriber identification module (SIM) 196, or an antenna module 197. In various examples, at least one of the components (e.g., the connection terminal 178) may be omitted from the electronic device 101, or one or more other components may be added in the electronic device 101. In various examples, some of the components (e.g., the sensor module 176, the camera module 180, or the antenna module 197) may be implemented as a single component (e.g., the display module 160).

The processor 120 may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions. The processor 120 may execute, for example, software (e.g., a program 140) to control at least one other component (e.g., a hardware or software component) of the electronic device 101 coupled with the processor 120, and may perform various data processing or computation. According to an example, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an example, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an example, the auxiliary processor 123 (e.g., an image signal processor or a communication processor) may be implemented as part of another component (e.g., the camera module 180 or the communication module 190) functionally related to the auxiliary processor 123. According to an example, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134. The non-volatile memory may include at least one of an internal memory 136 and an external memory 138.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an example, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an example, the display module 160 may include a touch sensor adapted to detect a touch, or a pressure sensor adapted to measure the intensity of force incurred by the touch.

The audio module 170 may convert a sound into an electrical signal and vice versa. According to an example, the audio module 170 may obtain the sound via the input module 150, or output the sound via the sound output module 155 or a headphone of an external electronic device (e.g., the electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an example, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an example, the interface 177 may include, for example, a high definition multimedia interface (HDMI), a universal serial bus (USB) interface, a secure digital (SD) card interface, or an audio interface.

The connection terminal 178 may include a connector via which the electronic device 101 may be physically connected with the external electronic device (e.g., the electronic device 102). According to an example, the connection terminal 178 may include, for example, an HDMI connector, a USB connector, an SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an example, the haptic module 179 may include, for example, a motor, a piezoelectric element, or an electric stimulator.

The camera module 180 may capture a still image or moving images. According to an example, the camera module 180 may include one or more lenses, image sensors, image signal processors, or flashes.

The power management module 188 may manage power supplied to the electronic device 101. According to one example, the power management module 188 may be implemented as at least part of, for example, a power management integrated circuit (PMIC).

The battery 189 may supply power to at least one component of the electronic device 101. According to an example, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an example, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a fifth generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a fourth generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the millimeter wave (mm Wave) band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an example, the wireless communication module 192 may support a peak data rate (e.g., 20 gigabits per second (Gbps) or more) for implementing eMBB, loss coverage (e.g., 164 decibels (dB) or less) for implementing mMTC, or U-plane latency (e.g., 0.5 milliseconds (ms) or less for each of downlink (DL) and uplink (UL), or a round trip of Ims or less) for implementing URLLC.

The antenna module 197 may transmit or receive a signal or power to or from the outside (e.g., the external electronic device) of the electronic device 101. According to an example, the antenna module 197 may include an antenna including a radiating element including a conductive material or a conductive pattern formed in or on a substrate (e.g., a printed circuit board (PCB)). According to an example, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an example, another component (e.g., a radio frequency integrated circuit (RFIC)) other than the radiating element may be additionally formed as part of the antenna module 197.

According to various examples, the antenna module 197 may form an mm Wave antenna module. According to an example, the mm Wave antenna module may include a printed circuit board, an RFIC disposed on a first surface (e.g., the bottom surface) of the printed circuit board, or adjacent to the first surface and capable of supporting a designated high-frequency band (e.g., the mm Wave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an example, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an example, all or some of operations to be executed at the electronic device 101 may be executed at one or more of the external electronic devices (e.g. electronic devices 102 and 104 or the server 108). For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In an example, the external electronic device 104 may include an internet-of-things (IoT) device. The server 108 may be an intelligent server using machine learning and/or a neural network. According to an example, the external electronic device 104 or the server 108 may be included in the second network 199. The electronic device 101 may be applied to intelligent services (e.g., smart home, smart city, smart car, or healthcare) based on 5G communication technology or IoT-related technology.

The electronic device according to various examples may be one of various types of electronic devices. The electronic devices may include, for example, a portable communication device (e.g., a smartphone), a computer device, a portable multimedia device, a portable medical device, a camera, a wearable device, or a home appliance. According to an example of the disclosure, the electronic devices are not limited to those described above.

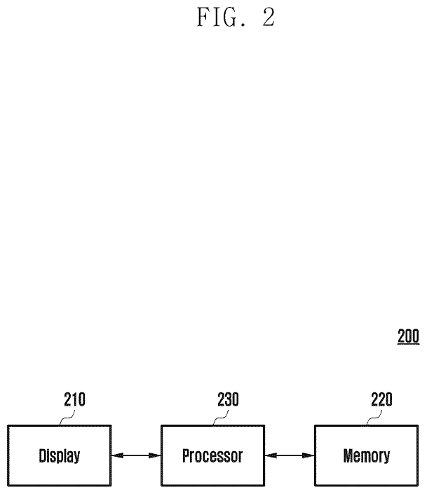

FIG. 2 is a block diagram illustrating an example configuration of an electronic device according to various embodiments.

With reference to FIG. 2, the electronic device 200 may include a display 210, a memory 220, and a processor (e.g., including processing circuitry) 230.

The display 210 (e.g., the display module 160 in FIG. 1) may output data processed by the processor 230 (e.g., the processor 120 in FIG. 1) as an image. For example, the display 210 may display a user interface (UI) for entering (or registering) a medication, a visual object including analysis data analyzed based on entered medication information and/or user's biometric information, etc.

For example, the analysis data may include a first factor, a first score according to the first factor, a second factor, and/or a second score according to the second factor. For example, the first factor may include a factor commonly applicable to a plurality of medication categories. In an example, the first factor may include activity information, sleep information, condition information, and/or calorie information. The first score according to the first factor may include a score that quantifies the first factor. For example, the second factor may include a factor applicable to a medication category related to medications. In an example, the second factor may include weight information, blood sugar information, blood pressure information, heart rate information, sleep information, and/or self-diagnosis information. The second score according to the second factor may include a score that quantifies the second factor. For example, the visual object may be an object that shows the analysis data in a visualized form for the convenience of the user. The visual object may include the first factor, the first score according to the first factor, the second factor, and/or the second score according to the second factor. The visual object may include a chart, a tree map, a heat map, an analysis table, a datasheet, a graph, and/or a histogram.

The memory 220 (e.g., the memory 130 in FIG. 1) may store data, algorithms, programs, instructions, etc. that perform the functions of the electronic device 200 (e.g., the electronic device 101 in FIG. 1). The instructions, etc. stored in the memory 220 can be loaded into the processor 230 and executed by the processor 230. In an example, the memory 220 may store information on a medication, a dosage schedule, a history of taking one or more medications, biometric information, self-diagnosis information entered by a user, analysis data, and/or a visual object.

The processor 230 can control each component of the electronic device 200. The electronic device 101 may include one or more processors 230. The one or more processors 230 may individually or collectively execute instructions. For example, the processor 230 may correspond to a plurality of processors that collectively perform a plurality of functions divided therefor. The processor 230 may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

For example, the processor 230 may receive information on a medication based on an input (e.g., a first user input). In an example, the processor 230 may receive information on a medication through an input of a medication name by a user, photographing (or scanning) of a medication name using a camera, and/or association with a prescription. The processor 230 may obtain a medication category related to the medication based on the received information on the medication. In an example, the processor 230 may classify the medication as a predetermined (e.g., specified) medication category based on the received information on the medication. In an example, an external device (e.g., a server or a database) may classify the medication category based on the information on the medication. The processor 230 may receive information about the medication category from the external device. The processor 230 may obtain classification information about the medication from a database connected through an external device (e.g., a server), or obtain a medication category based on the information on the medication.

For example, the medication category may include a first medication category and a second medication category. The first medication category may be a medication category in which analysis data obtained after taking the medication contains qualitative information (e.g., self-diagnosis information), and the second medication category may be a medication category in which the analysis data contains quantitative information. In an example, if the category of the medication taken is related to pain relief, the analysis data may contain qualitative information including the degree of pain symptom relief, so the category related to pain relief may be classified as the first medication category. If the category of the medication taken is related to weight loss, the analysis data may contain weight loss information, so the medication category related to weight loss may be classified as the second medication category. The medications classified as the first medication category or the second medication category may be classified into detailed categories according to the type of medication and/or the corresponding symptom. In an example, a medication related to pain relief may be classified as the first medication category and an analgesic category. A medication related to weight loss may be classified as the second medication category and a diet medication category.

In an example, the information on the medication may include a dosage schedule. The processor 230 may provide a dosage reminder to the user, based on the dosage schedule. For example, a dosage reminder UI may contain an item for entering whether the medication is taken. The processor 230 may receive a dosage completion input (e.g., a second user input) from the user. For example, the processor 230 may receive the dosage completion input containing a time when the medication was taken, entered directly by the user. For example, the processor 230 may regard, as time information when the medication was taken, current time information of the electronic device 200 when receiving the dosage completion input from the user. The dosage completion input received from the user may indicate the time information when the medication was taken.

The processor 230 may obtain user's biometric information. For example, the processor 230 may continuously and/or sequentially obtain user's biometric information at a certain period or at a predetermined time. Based on the dosage schedule of the medication, the processor 230 may determine whether the obtained biometric information is biometric information before taking the medication or biometric information after taking the medication.

Based on the obtained biometric information and the time information when the medication was taken, the processor 230 may obtain the first score according to the first factor commonly applicable to a plurality of medication categories, and the second score according to the second factor applicable to a medication category related to the taken medication. For example, the first factor may include activity information, sleep information, condition information, and/or calorie information. The score according to the first factor may include a score calculated based on the first factor. For example, the second factor may include weight information, blood sugar information, blood pressure information, heart rate information, sleep information, and/or self-diagnosis information. The score according to the second factor may include a score calculated based on the second factor. The processor 230 may provide the visual object representing the first score according to the first factor and the second score according to the second factor through the display 210. For example, the visual object may include a chart, a tree map, a heat map, an analysis table, a datasheet, a graph, and/or a histogram.

The effect of the medication may occur after a certain period of time from a dosage time of the medication. The processor 230 may determine an effect onset time, based on the medication and/or the dosage schedule. For example, if the medication is classified as the first medication category, the processor 230 may receive self-diagnosis information from the user. In an example, in the case that the medication is classified as the first medication category, the processor 230 may determine the effect onset time from the dosage time of the medication. If the effect onset time is satisfied, the processor 230 may receive the self-diagnosis information from the user. The processor 230 may provide the visual object including the self-diagnosis information as the second factor.

For example, in relation to a medication for short-term use (e.g., a painkiller), the electronic device 200 may calculate the first score according to the first factor and the second score according to the second factor, based on the biometric information obtained after the effect onset time (e.g., 2 hours after the dosage time). The electronic device 200 may generate the visual object, based on the calculated first and second scores. For example, the score at the effect onset time (or the score after the effect onset time) contained in the visual object may be the user's most recent update score identified at the effect onset time (or after the effect onset time).

For example, in relation to a medication for continuous use (e.g., a sleeping pill), the electronic device 200 may calculate the first score according to the first factor and the second score according to the second factor, based on the biometric information obtained periodically from the time of starting taking the medication (e.g., the day of starting taking the medication). The electronic device 200 may average each of the calculated first score and second score according to a regular cycle (e.g., daily) and generate the visual object based on the calculated average value. For example, the first score according to the first factor and the second score according to the second factor may be updated at a regular cycle (e.g., daily). In other words, the electronic device 200 may obtain the first score and the second score that are recently updated based on the user's biometric information acquired after the effect onset time or dosage time of the taken medication.

The second factor may include different information depending on the medication category related to the taken medication. For example, if the medication category of the taken medication is related to pain relief, the processor 230 may receive the self-diagnosis information including the degree of pain symptom relief from the user. The processor 230 may provide the received self-diagnosis information as the second score according to the second factor. For example, if the medication category of the taken medication is related to weight loss, the processor 230 may determine reduced weight based on the user's biometric information. The processor 230 may provide weight information including the reduced weight as the second score according to the second factor. For example, if the medication category of the taken medication is related to insomnia, the processor 230 may determine a deep sleep time based on the user's biometric information. The processor 230 may provide sleep information including the deep sleep time as the second factor. For example, if the medication category of the taken medication is related to blood pressure, the processor 230 may determine blood pressure information based on the user's biometric information. The processor 230 may provide the blood pressure information as the second score according to the second factor. For example, if the medication category of the taken medication is related to diabetes, the processor 230 may determine blood sugar information based on the user's biometric information. The processor 230 may provide the blood sugar information as the second score according to the second factor. For example, if the medication category of the taken medication is related to arrhythmia, the processor 230 may determine the regularity of the pulse based on the user's biometric information. The processor 230 may provide heart rate information including the regularity of the pulse as the second score according to the second factor.

For example, the processor 230 may store a history of medications previously taken by the user and analysis data of the taken medications in the memory 220. In response to a user's request, the processor 230 may determine related medications included in the same medication category (or corresponding to the same type or same symptom) as the currently taken medication, and identify analysis data of the related medications. The processor 230 may provide the analysis data of one or more related medications to the user. For example, the processor 230 may determine the effect of the medication from the first score according to the first factor and/or the second score according to the second factor included in the analysis data. The processor 230 may receive information on side effects from the user. The processor 230 may determine the side effect of the medication from the received information on side effects. The processor 230 may generate the visual object representing the analysis data, based on the effect and/or side effect of the medication, and then provide the generated visual object to the user through the display 210.

For example, the processor 230 may receive the analysis data of related medication taken by other person through a communication interface (e.g., the communication module 190 in FIG. 1). The other person may include a family member or another person with a predetermined correlation or higher. In an example, the predetermined correlation may include a correlation of overall biometric information between the user and the other person, a correlation of biometric information related to symptoms between the user and the other person, a correlation of the first score according to the first factor and the second score according to the second factor between the user and the other person, and/or a correlation of side effects between the user and the other person. The received analysis data may include information on the effect and/or side effect of the medication. The processor 230 may generate the visual object representing the analysis data of related medication taken by the other person, based on the effect and/or side effect of the medication, and then provide the generated visual object to the user through the display 210).

For example, the processor 230 may provide the information on a medication included in a medication category according to a symptom entered by the user, based on the effect and/or side effect using medication analysis data learned through an artificial intelligence model. The processor 230 may provide information on a single medication and information on a combination of multiple medications. In an example, if the combination of multiple medications includes a main medication and a sub medication, the processor 230 may provide information on the combination of multiple medications by discriminatively representing the main medication and the sub medication. In an example, the processor 230 may use different colors, line thicknesses, and/or sizes to discriminate the main medication and the sub medication.

For example, the processor 230 may receive information about a side effect from the user and store biometric information related to the side effect in the memory 220. In an example, the processor 230 may periodically obtain biometric information including heart rate information. If the obtained biometric information of the user is within a predetermined range with the biometric information related to the side effect, the processor 230 may provide a notification to the user. In an example, if the heart rate of the user increases from about 80 to about 100 after taking a medication, the processor 230 may store biometric information including the heart rate information increased to about 100. If the predetermined range is 5%, the processor 230 may provide a notification to the user when the heart rate becomes 95 or higher.

The electronic device, method, and storage medium of the disclosure can check the effect of a medication taken, its efficacy, and its impact on health indexes by associating with biometric information. The electronic device, method, and storage medium of the disclosure can provide information on other medications based on effects and/or side effects.

The effects of the disclosure are not limited to those mentioned above, and other effects not mentioned will be clearly understood by those skilled in the art from the description below.

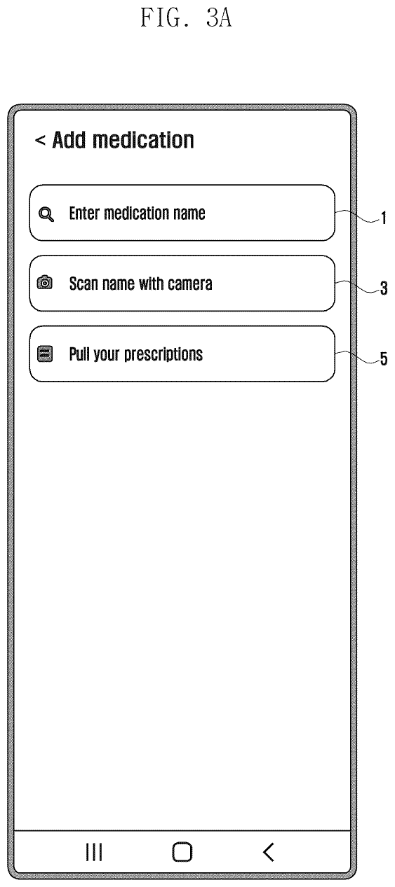

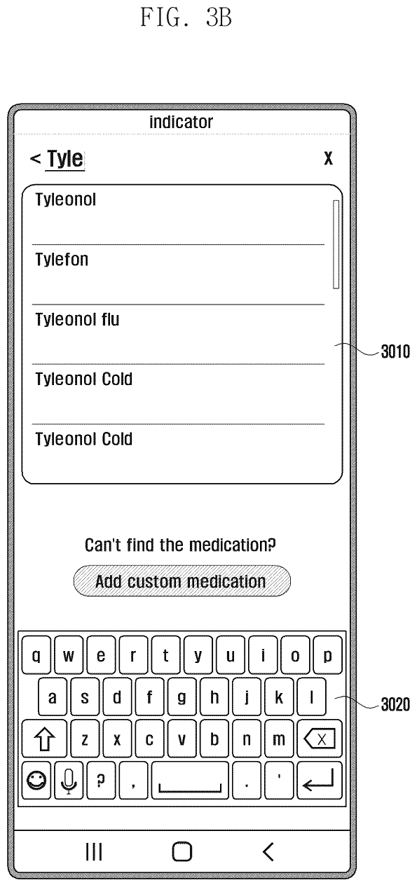

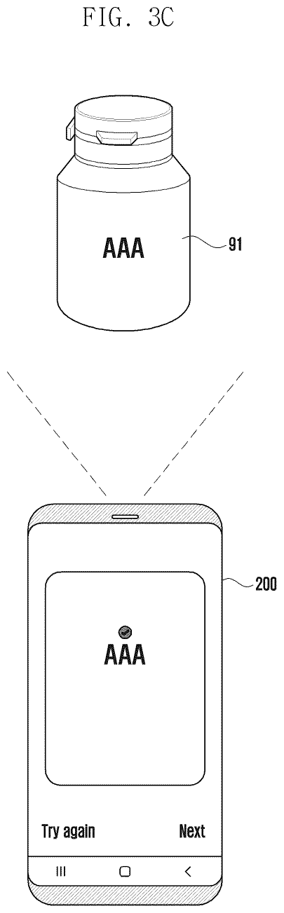

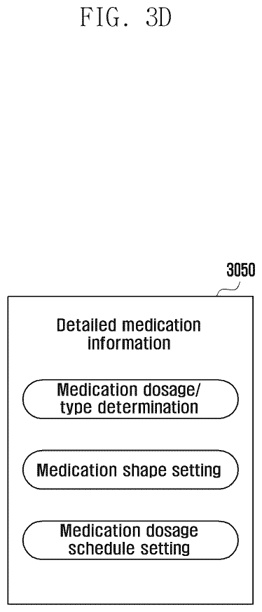

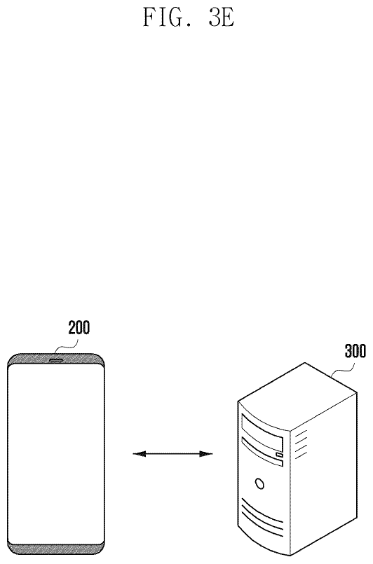

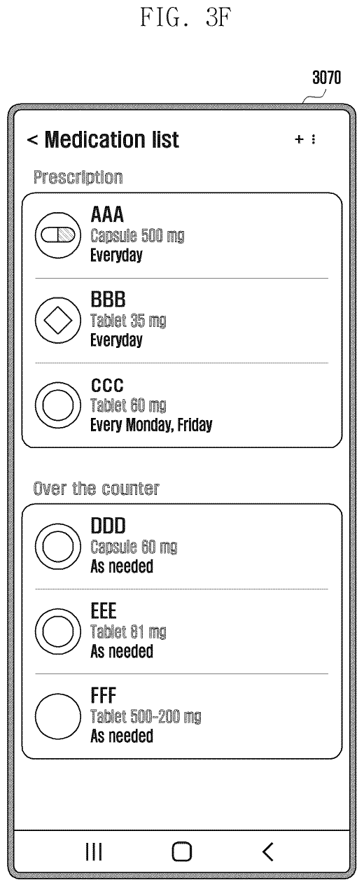

FIGS. 3A, 3B, 3C, 3D, 3E, and 3F are diagrams illustrating an example operation of adding information on a taken medication according to various embodiments.

With reference to FIG. 3A, a UI for adding information on a taken medication is illustrated. The electronic device 200 may display this UI in response an input (e.g., a user's input). The UI for adding the medication information may include a medication name input item 1, a medication name scan item 3 using a camera, and a prescription input item 5.

With reference to FIG. 3B, a UI for entering a medication name is illustrated. When the medication name input item 1 is selected in FIG. 3A, the electronic device 200 may display the UI for entering a medication name. For example, a first area 3010 of the UI may display a list of medication names related to input characters (or including numbers), and a second area 3020 may display a character input area. For example, the input area may be implemented as a keyboard, a keypad, or a writing area. The electronic device 200 may display, in the first area 3010, a list of medication names having characters entered in the input area. For example, the list of medication names may be stored in the memory 220 or an external device (e.g., the electronic device 102 or the server 108 in FIG. 1). Based on the input characters, the electronic device 200 may load (or receive) medication names from the memory 220 or the external device and display them in the first area 3010. When one medication name from the list of medication names is selected, the electronic device 200 may receive information on a medication corresponding to the selected medication name from the external device.

With reference to FIG. 3C, a camera that photographs a medication bottle 91 is illustrated. When the medication name scan item 3 using a camera is selected in FIG. 3A, the electronic device 200 may drive the camera (e.g., the camera module 180 in FIG. 1). The medication bottle 91 may have a medication name printed thereon. The electronic device 200 may recognize the medication name printed on the medication bottle 91. For example, the electronic device 200 may recognize the medication name from an image obtained by photographing the medication name on the medication bottle 91 or recognize the medication name from a preview image containing the medication name. Upon recognizing the medication name, the electronic device 200 may receive information on a medication corresponding to the recognized medication name from the external device.

With reference to FIG. 3D, a UI 3050 for entering detailed medication information is illustrated. The medication information received from the external device may include only general information. Even for the same medication, a method of taking the medication may vary depending on the patient's status and/or the degree of symptoms. When medication information is received by entering the medication name or scanning the medication name using the camera, the electronic device 200 may display the UI 3050 for entering detailed medication information. For example, this UI 3050 may contain a medication dosage/type determination item, a medication shape setting item, and a dosage schedule setting item. The electronic device 200 may receive detailed medication information including a dosage schedule, etc. from the user on the UI 3050 for entering detailed medication information.

With reference to FIG. 3E, the electronic device 200 that receives information on a medication in connection with a prescription is illustrated. When the prescription input item 5 is selected in FIG. 3A, the electronic device 200 may communicate with an external device 300 that includes information on a prescription. The electronic device 200 may receive the information on the medication from the information on the prescription included in the external device 300. The information on the prescription may include information about a medication dosage and/or a dosage schedule, etc. The electronic device 200 may receive detailed medication information including the dosage schedule, etc. as well as general medication information from the information on the prescription.

With reference to FIG. 3F, a UI 3070 for showing a list of input medications is illustrated. For example, the input medication list may include information on a medication name, a medication type, a medication shape, a medication dosage, and/or a dosage schedule. The electronic device 200 may classify the input medications into a plurality of groups according to a predetermined criterion and discriminatively display the respective medication groups on the UI 3070 that shows the medication list. For example, the electronic device 200 may classify the input medications into a group of medications taken periodically and a group of medications taken regularly when necessary. The electronic device 200 may discriminatively display the group of medications taken periodically and the group of medications taken regularly on the UI 3070 that shows the medication list.

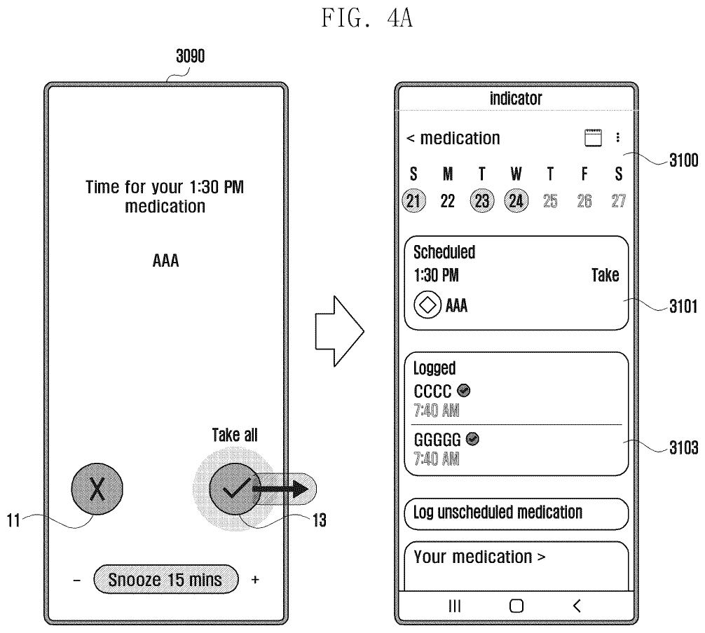

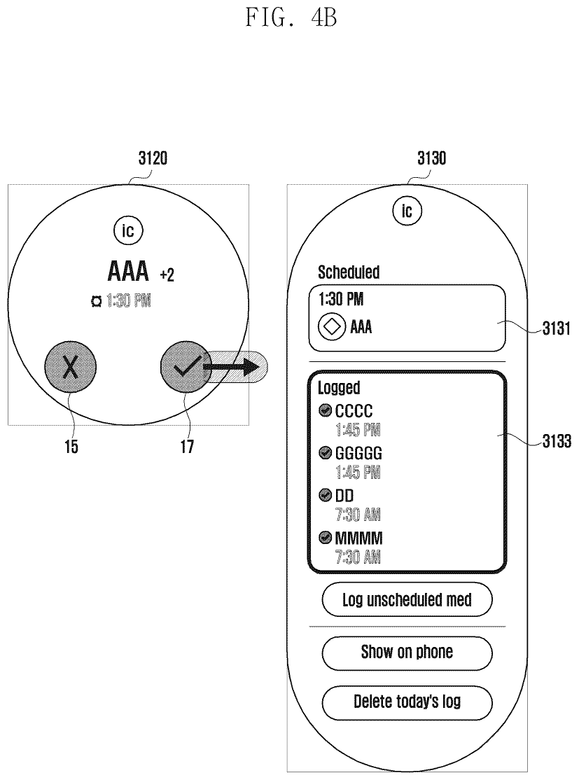

FIGS. 4A and 4B are diagrams illustrating an example operation of managing a schedule for taking a medication according to various embodiments.

With reference to FIG. 4A, a dosage reminder UI 3090 and a dosage completion UI 3100 are illustrated. The electronic device 200 may provide a dosage reminder to the user, based on an input dosage schedule. For example, if a dosage schedule for AAA medication is 1:30 PM, the electronic device 200 may provide a dosage reminder for AAA medication at 1:30 PM. The electronic device 200 may display the dosage reminder UI 3090 and output a reminder sound and/or vibration. For example, the dosage reminder UI 3090 may contain information on a dosage time and/or a medication. In addition, the dosage reminder UI 3090 may contain a dosage-uncompleted input item 11 and a dosage completion input item 13. For example, if the dosage-uncompleted input item 11 is selected, the electronic device 200 may determine that the user has not taken the medication. If the dosage completion input item 13 is selected, the electronic device 200 may determine that the user has taken the medication, and then display the dosage history UI 3100. For example, the electronic device 200 may receive the medication-taken time information directly from the user along with the selection of the dosage completion input item 13. For example, when the dosage completion input item 13 is selected by the user, the electronic device 200 may determine the current time information of the electronic device 200 as the medication-taken time information. The dosage history UI 3100 may include a history of medications that have been taken. For example, the dosage history UI 3100 may include an area 3101 having the dosage completion information of current dosing target medications and/or an area 3103 having the dosage completion information of previous dosing target medications. In an example, these areas 3101 and 3013 having the dosage completion information may include information on taken medications and/or the time of taking the medications.

With reference to FIG. 4B, a dosage reminder UI 3120 and a dosage completion UI 3130 displayed on a wearable device are illustrated. For example, the electronic device 200 may be a wearable device. The wearable device may include, for example, and without limitation, a smart watch and/or a smart ring.

Even if the electronic device 200 is a wearable device, the electronic device 200 may perform an operation similar to the operation described in FIG. 4A. For example, the electronic device 200 may provide a dosage reminder to the user, based on an input dosage schedule, and display a dosage reminder UI 3120. The dosage reminder UI 3120 may contain a dosage-uncompleted input item 15 and a dosage completion input item 17. For example, if the dosage-uncompleted input item 15 is selected, the electronic device 200 may determine that the user has not taken the medication. If the dosage completion input item 17 is selected, the electronic device 200 may determine that the user has taken the medication, and then display a dosage history UI 3130. For example, the dosage history UI 3130 may include an area 3131 having the dosage completion information of current dosing target medications and/or an area 3133 having the dosage completion information of previous dosing target medications.

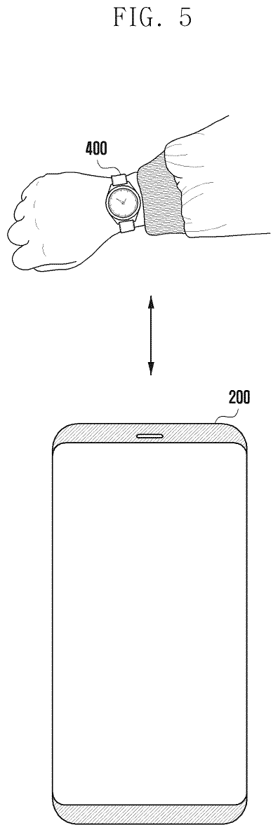

FIG. 5 is a diagram illustrating an example system for receiving biometric information from a wearable device according to various embodiments.

With reference to FIG. 5, for example, the electronic device 200 may be implemented as a smartphone. In this case, the electronic device 200 includes a communication interface (e.g., the communication module 190 in FIG. 1) and may receive biometric information (or health data) from the wearable device 400. For example, the wearable device 400 may be worn on the user's body. The wearable device 400 may include a sensor (e.g., the sensor module 176 in FIG. 1) and may obtain biometric information of the user. For example, the biometric information may include weight information, blood sugar information, blood pressure information, heart rate information, and/or sleep information. The wearable device 400 may transmit the obtained biometric information to the electronic device 200 through a communication interface (e.g., the communication module 190 in FIG. 1). The electronic device 200 may generate analysis data including the first score according to the first factor and the second score according to the second factor based on the received biometric information and may generate the visual object representing the generated analysis data. The electronic device 200 may provide the generated visual object to the user. For example, the visual object may include a chart, a tree map, a heat map, an analysis table, a datasheet, a graph, and/or a histogram. In an example, the wearable device 400 may generate analysis data based on the obtained biometric information. The wearable device 400 may transmit the generated analysis data to the electronic device 200 through the communication interface (e.g., the communication module 190 in FIG. 1). The electronic device 200 may generate the visual object representing the received analysis data. The electronic device 200 may provide the generated visual object to the user. In an example, the wearable device 400 may generate the analysis data based on the obtained biometric information and generate the visual object representing the analysis data. The wearable device 400 may transmit the generated visual object to the electronic device 200 through the communication interface (e.g., the communication module 190 in FIG. 1). The electronic device 200 may provide the received visual object to the user.

For example, an electronic device may be implemented as the wearable device 400. In this case, the electronic device may obtain user's biometric information. In an example, the wearable device 400 may transmit the obtained user's biometric information to an external device (e.g., the electronic device 200 in FIG. 5) via a communication interface (e.g., the communication module 190 in FIG. 1). The external device that receives the user's biometric information may generate analysis data and/or a visual object representing the analysis data and provide the generated visual object to the user. In an example, the wearable device 400 may generate the analysis data based on the obtained user's biometric information. The wearable device 400 may transmit the generated analysis data to the external device through the communication interface (e.g., the communication module 190 in FIG. 1). The external device that receives the analysis data may generate the visual object representing the analysis data and provide the generated visual object to the user. In an example, the wearable device 400 may generate the analysis data and/or the visual object representing the analysis data based on the obtained user's biometric information. The wearable device 400 may transmit the generated visual object to the external device through the communication interface (e.g., the communication module 190 in FIG. 1). The external device that receives the visual object may provide the received visual object to the user. For example, if the number of analysis data and/or visual objects is greater than or equal to a predetermined number, the wearable device 400 may transmit the analysis data and/or visual objects to the external device. If there is a lot of information related to the analysis data and/or visual objects, the wearable device 400 may transmit the analysis data and/or visual objects to the external device so that the external device may display them. In an example, the wearable device 400 may transmit the analysis data and/or visual objects containing different information depending on the information and/or dosage of different medications to the external device.

For example, in the case that the electronic device 200 is implemented as a smartphone, the electronic device 200 may include a sensor. When the electronic device 200 includes the sensor, the electronic device 200 may obtain user's biometric information using the sensor. In an example, the electronic device 200 may provide a notification for measuring biometric information to the user at a predetermined time and/or according to a certain cycle. When the sensor comes into contact with the user's body, the electronic device 200 may obtain biometric information. The electronic device 200 may generate analysis data based on the obtained biometric information and generate a visual object based on the generated analysis data.

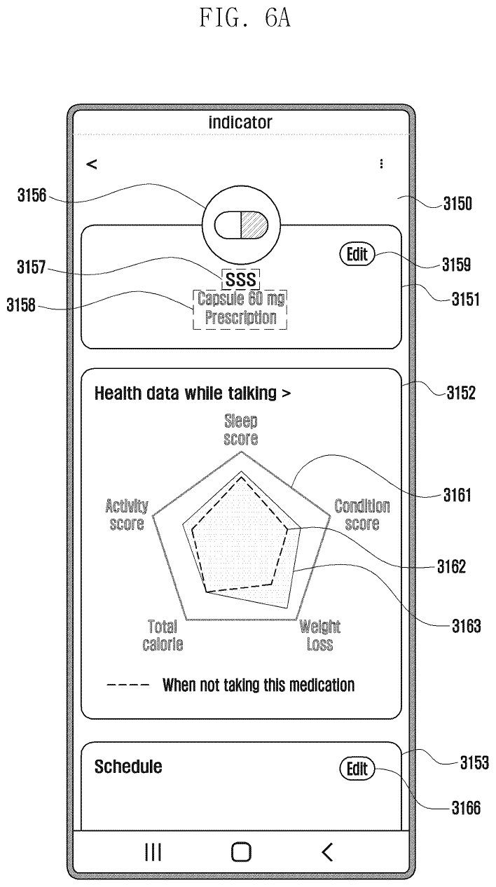

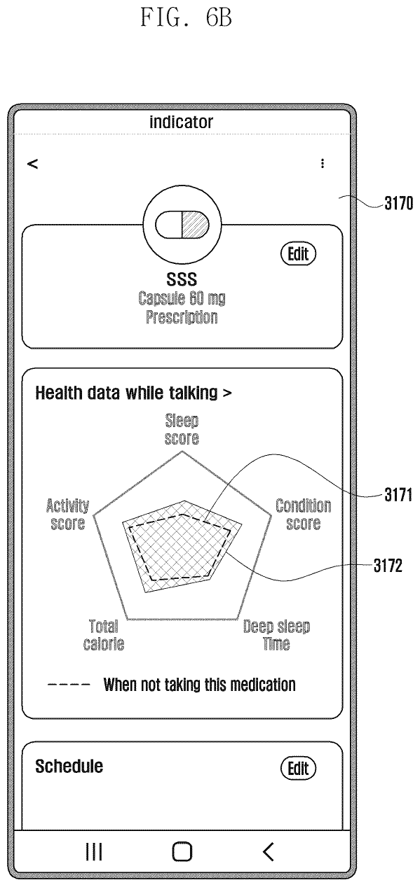

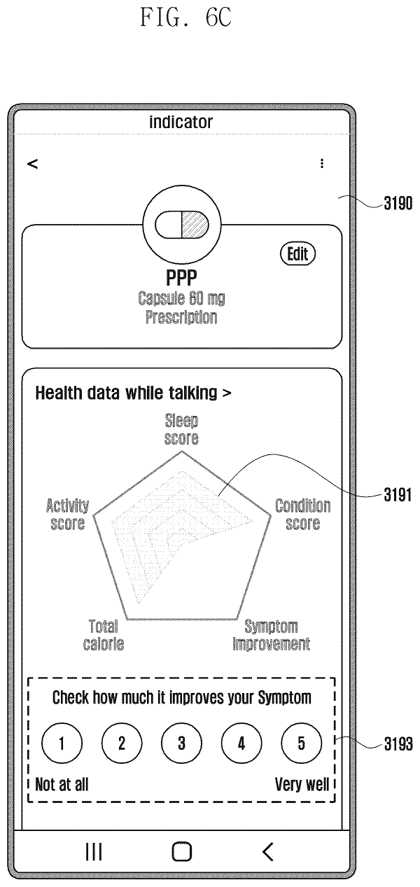

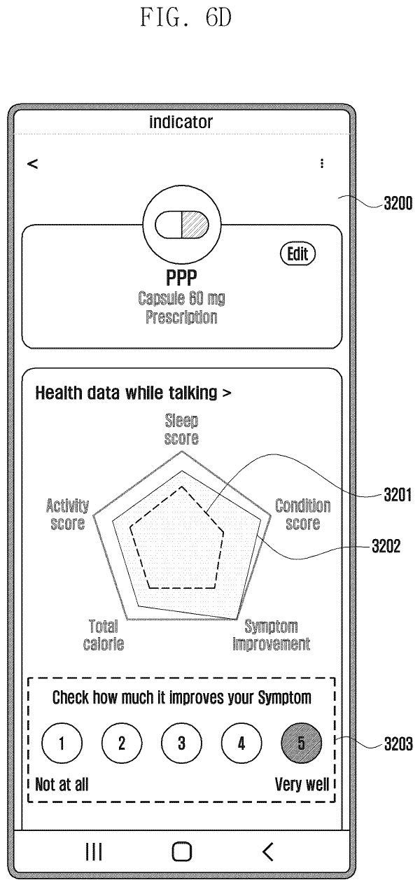

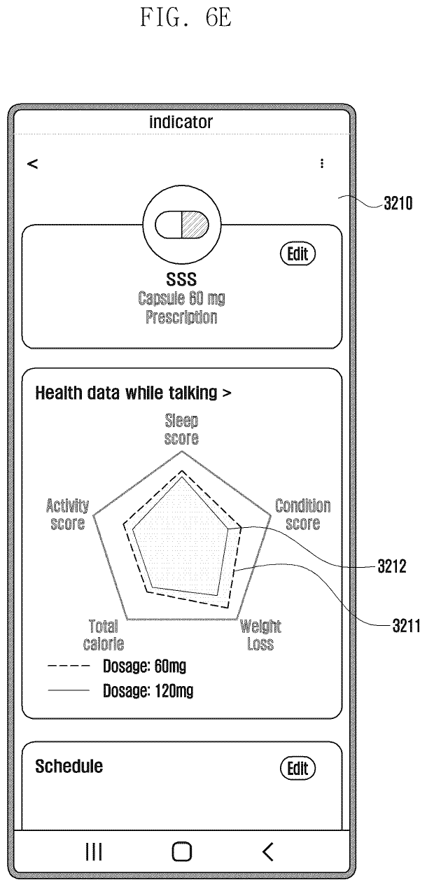

FIGS. 6A, 6B, 6C, 6D, and 6E are diagrams illustrating example UIs of analysis data according to various embodiments.

For example, the analysis data may include a first factor commonly applicable to a plurality of medication categories, a first score according to the first factor, the second factor applicable to a medication category related to a medication, and a second score according to the second factor. In an example, the first factor may include activity information, sleep information, condition information, and/or calorie information, and the second factor may include weight information, blood sugar information, blood pressure information, heart rate information, sleep information, and/or self-diagnosis information. For example, the activity information may indicate a user's activity status. For example, the activity information may include the number of steps, the amount of exercise, and/or the type of exercise. An activity score may be an indicator that quantifies the user's activity status. The activity score may be calculated based on activity calories according to the number of steps and the amount of exercise, and a weight according to the intensity of exercise. The sleep information may indicate a user's sleep quality. For example, the sleep information may include total sleep time, sleep regularity, sleep start time, percentage of deep sleep, percentage of wakes during sleep, percentage of REM sleep, number of sleep cycles, sleep heart rate, and/or sleep heart rate variability. A sleep score may be an indicator that quantifies the user's sleep quality. The sleep score may be calculated based on the total sleep time, percentage of deep sleep, percentage of wakes during sleep, percentage of REM sleep, and/or number of sleep cycles. The condition information may indicate a condition (e.g., energy or vitality) at the start of the day, and a condition score may be referred to as an energy score or a vitality score. The condition score may be an indicator that quantifies the condition at the start of the day. The condition score may be calculated based on the sleep information and activity information of the previous day. The calorie information may indicate the intake and consumption of calories. For example, the calorie information may include the intake of calories compared to recommended calories, basal metabolic rate, and/or calories consumed. A calorie score may be an indicator indicating the intake and consumption of calories. The calorie score may be calculated based on the intake of calories compared to recommended calories, basal metabolic rate, and/or calories consumed. The calorie score may be provided as a score or provided as an indicator in the form of calorie balance.

With reference to FIG. 6A, a UI 3150 of analysis data of a weight loss medication is illustrated. This UI 3150 may contain an information area 3151 of the taken weight loss medication, a visual object area 3152 representing analysis data related to taking the weight loss medication, and a dosage schedule area 3153. In an example, the information area 3151 of the weight loss medication may include an image (or icon) 3156 of the weight loss medication, a name 3157 of the weight loss medication, a dosage 3158, and a medication information edit item 3159. For example, the medication information edit item 3159 allows modifying the image (or icon) 3156 of the weight loss medication, the name 3157 of the weight loss medication, and/or the dosage 3158. In an example, the visual object area 3152 may include a base area 3161 of a visual object, a pre-taking visual object 3162 that indicates the first and second scores before taking the medication, and a post-taking visual object 3163 that indicates the first and second scores after taking the medication. For example, a first factor of the analysis data of the weight loss medication may include activity information, sleep information, condition information, and/or calorie information. A second factor may include weight information including reduced weight. In an example, the base area 3161 of the visual object may be depicted in the form of a figure connecting the maximum value of the first score according to the first factor and the maximum value of the second score according to the second factor. Each of the pre-taking visual object 3162 and the post-taking visual object 3163 may be depicted in the form of a figure connecting the first score and the second score. The electronic device 200 may provide the pre-taking visual object 3162 including the first and second scores and the post-taking visual object 3163 including the first and second scores as health indicators related to taking the weight loss medication. In an example, the dosage schedule area 3153 includes a dosage schedule related to the currently displayed medication and may include a dosage schedule edit item 3166 that allows editing the dosage schedule.

With reference to FIG. 6B, a UI 3170 of analysis data of an insomnia medication is illustrated. This UI 3170 may contain an information area of the insomnia medication and a visual object area for analysis data related to taking the insomnia medication. For example, the visual object area of the insomnia medication may include a pre-taking visual object 3171 that indicates the first and second scores before taking the medication, and a post-taking visual object 3172 that indicates the first and second scores after taking the medication. For example, a first factor of the analysis data of the insomnia medication may include activity information, sleep information, condition information, and/or calorie information. A second factor may include sleep information including a deep sleep time. The electronic device 200 may provide the pre-taking visual object 3171 including the first and second scores and the post-taking visual object 3172 including the first and second scores as health indicators related to taking the insomnia medication.

With reference to FIG. 6C, a UI 3190 of analysis data of a medication related to pain relief, excluding a score according to a second factor, is illustrated. For example, the second factor of the analysis data of the pain relief related medication may include self-diagnosis information including the degree of pain symptom relief. A first factor of the pain relief related medication may include activity information, sleep information, condition information, and/or calorie information. The self-diagnosis information may be qualitative information received from the user. For example, if the self-diagnosis information is not received, the electronic device 200 may provide a post-taking visual object 3191 including the first score according to the first factor as a health indicator related to taking the pain relief medication, omitting the second score according to the second factor and a pre-taking visual object. For example, the UI 3190 of the analysis data of the pain relief related medication may include an input area 3193 for entering the self-diagnosis information. The electronic device 200 may receive the self-diagnosis information through the input area 3193 after an effect onset time of the taken medication has passed.

With reference to FIG. 6D, a UI 3200 of analysis data of the pain relief related medication, including the second score according to the second factor, is illustrated. The electronic device 200 may receive self-diagnosis information through an input area 3203 for entering the self-diagnosis information in the UI 3200 of the analysis data of the pain relief related medication. Upon receiving the self-diagnosis information, the electronic device 200 may provide a pre-taking visual object 3201 including the first and second scores and a post-taking visual object 3202 including the first and second scores as health indicators related to taking the pain relief related medication.

In an example, if a taken medication is related to blood pressure, the electronic device 200 may provide before-taking and after-taking visual objects of analysis data including blood pressure information as the second factor. If a taken medication is related to diabetes, the electronic device 200 may provide before-taking and after-taking visual objects of analysis data including blood sugar information as the second factor. If a taken medication is related to arrhythmia, the electronic device 200 may provide before-taking and after-taking visual objects of analysis data including heartbeat information including pulse regularity as the second factor.

With reference to FIG. 6E, a UI 3210 of analysis data according to dosage is illustrated. The electronic device 200 may provide a plurality of visual objects 3211 and 3212 including the first score according to the first factor and the second score according to the second factor, based on dosage. In an example, as shown in FIG. 6E, the electronic device 200 may provide a first visual object 3211 including the first and second scores when a 60 mg weight loss medication is taken, and a first visual object 3212 including the first and second scores when a 120 mg weight loss medication is taken.

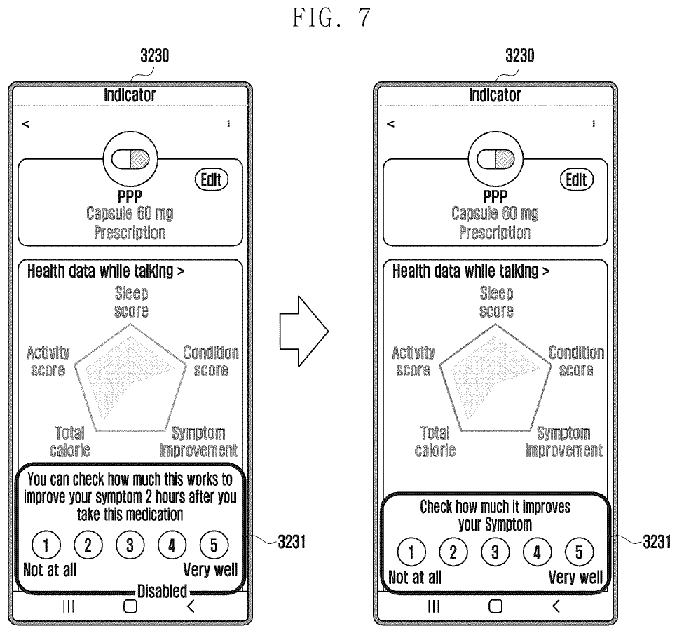

FIG. 7 is a diagram illustrating example UIs before and after an effect onset time of a taken medication according to various embodiments.

For example, the second factor of a medication classified as a first medication category may be self-diagnosis information. The self-diagnosis information may be a qualitative indicator of the effect felt by the user after taking the medication. The electronic device 200 may display an input area 3231 for entering the self-diagnosis information in a UI 3230 of analysis data of a medication classified as the first medication category (e.g., a medication related to pain relief). Because the medication requires time to take effect after taken, the electronic device 200 may receive the self-diagnosis information from the user after an effect onset time of the taken medication.

For example, if the medication is classified as the first medication category, the electronic device 200 may determine the effect onset time from a dosage time of the medication. In an example, if the effect onset time of the taken medication is two hours, the electronic device 200 may deactivate the input area 3231 for entering self-diagnosis information until two hours after taking the medication. If the input area 3231 for entering self-diagnosis information is in the deactivated state, the electronic device 200 cannot receive the self-diagnosis information, and thus, as described in FIG. 6C, may display the visual object excluding the second score according to the self-diagnosis information. If two hours have passed since the time of taking the medication, the electronic device 200 may activate the input area 3231 for entering self-diagnosis information. If the input area 3231 for entering self-diagnosis information is in the activated state, the electronic device 200 may receive the self-diagnosis information. Upon receiving the self-diagnosis information, the electronic device 200 may display the visual object including the second score according to the self-diagnosis information, as described above with reference to FIG. 6D.

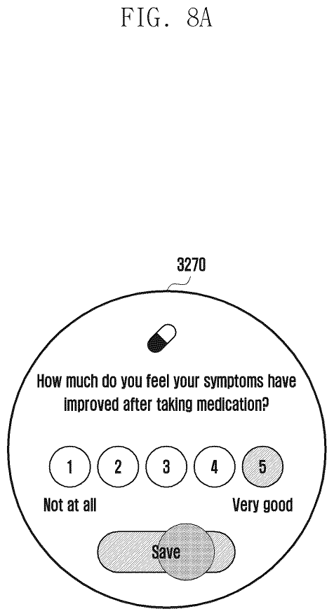

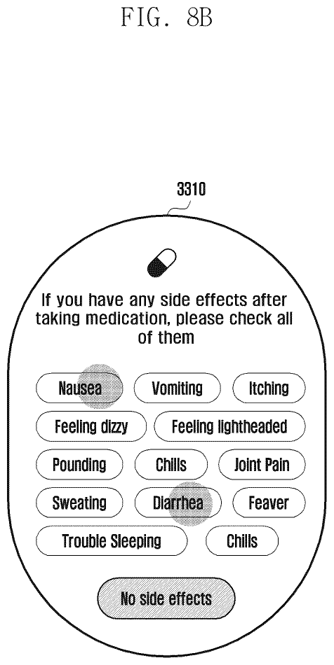

FIGS. 8A and 8B are diagrams illustrating an example operation of receiving information from a user according to various embodiments.

With reference to FIG. 8A, a self-diagnosis input UI 3270 is illustrated. For example, the electronic device 200 may display an area for entering self-diagnosis information in a UI of analysis data of a medication classified as a first medication category (e.g., a medication related to pain relief) and receive self-diagnosis information through the area for entering self-diagnosis information.

In an example, the electronic device 200 may display a separate self-diagnosis input UI 3270 to receive self-diagnosis information from the user. As described above, the medication may require time to take effect after taken. The electronic device 200 may determine an effect onset time of the taken medication. When the effect onset time of the taken medication has passed, the electronic device 200 may provide a notification to the user to guide the input of self-diagnosis information and display the self-diagnosis input UI 3270. For example, the electronic device 200 may display multiple stages for the degree of symptom improvement after taking the medication and receive an input for selecting one stage. In an example, the degree of symptom improvement may be divided into five stages from stage 1 of no effect to stage 5 of very good based on the degree of effect. Upon receiving a user's input of selecting and saving one stage, the electronic device 200 may include the self-diagnosis information of the taken medication as a second factor in the analysis data and display a visual object including a second score according to the second factor.

With reference to FIG. 8B, a side effect input UI 3310 is illustrated. The electronic device 200 may receive side effect information when receiving self-diagnosis information. In an example, the electronic device 200 may receive side effect information separately from receiving self-diagnosis information. For example, upon receiving an input of selecting and saving one stage on the self-diagnosis input UI 3270 shown in FIG. 8A, the electronic device 200 may display the side effect input UI 3310 shown in FIG. 8B. In an example, the electronic device 200 may display the side effect input UI 3310 at the user's request. The electronic device 200 may identify side effect keywords included in medication database and display them on the side effect input UI 3310. In an example, the medication database may be included in an external device, and the electronic device 200 may receive side effect keywords from the external device. When receiving an input of selecting and saving at least one side effect on the side effect UI 3310, the electronic device 200 may display information on the selected side effect together with a visual object.

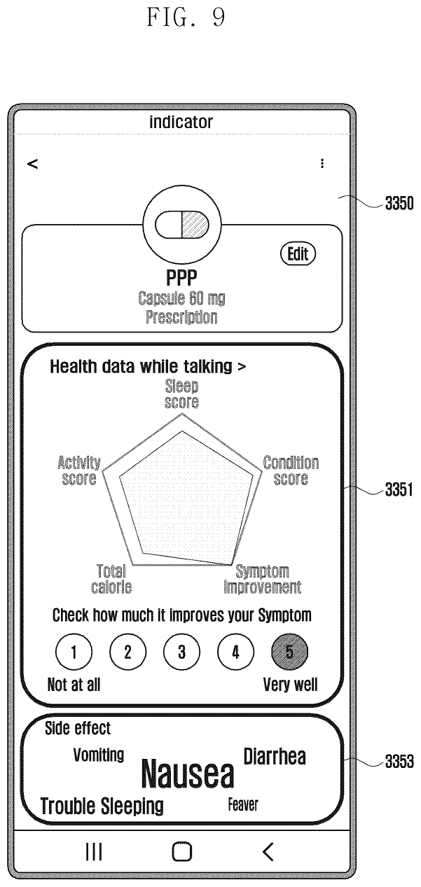

FIG. 9 is a diagram illustrating an example UI of analysis data including side effect information according to various embodiments.

With reference to FIG. 9, a UI 3350 of analysis data of a medication is illustrated. For example, the UI of the analysis data may contain a visual object area 3351 and a side effect information area 3353. The visual object area 3351 may include a first visual object including a first score according to a first factor and a second score according to a second factor before taking the medication, and a second visual object including the first score according to the first factor and the second score according to the second factor after taking the medication. For example, the side effect information area 3353 may include all side effect information received from the user. The electronic device 200 may display the side effect information differently according to the frequency and/or reception time of the received side effect information. For example, the electronic device 200 may discriminate side effect information by color, font size, font boldness, and/or shading. In an example, the electronic device 200 may display the most frequently received side effect information in the largest size. The electronic device 200 may display the most recently received side effect information in bold. In an example, the electronic device 200 may assign weights to the received side effect information according to the frequency and reception time, display side effect information with the highest weight in red, and display side effect information in other colors based on the weights.

For example, the electronic device 200 may store biometric information related to the received side effect information. The electronic device 200 may continuously obtain the biometric information periodically and/or at a predetermined time. If the obtained biometric information is within a predefined range compared to biometric information related to the side effect information, the electronic device 200 may provide a notification to the user. In an example, if the electronic device 200 receives side effect information that a fever is felt after taking medication, the electronic device 200 may obtain abnormal body temperature information at the time when the side effect information is received. In another example, the electronic device 200 may check abnormal body temperature information from biometric information obtained after an effect onset time after taking medication. In an example, if the abnormal body temperature information is 37 degrees, the electronic device 200 may store abnormal body temperature information of 37 degrees in conjunction with the received side effect information that a fever is felt. The electronic device 200 may continuously acquire body temperature information periodically or at a predetermined time. If the predefined range is 0.2 degrees and the body temperature information obtained by the electronic device 200 is 36.8 degrees, the electronic device 200 may provide a notification to the user.

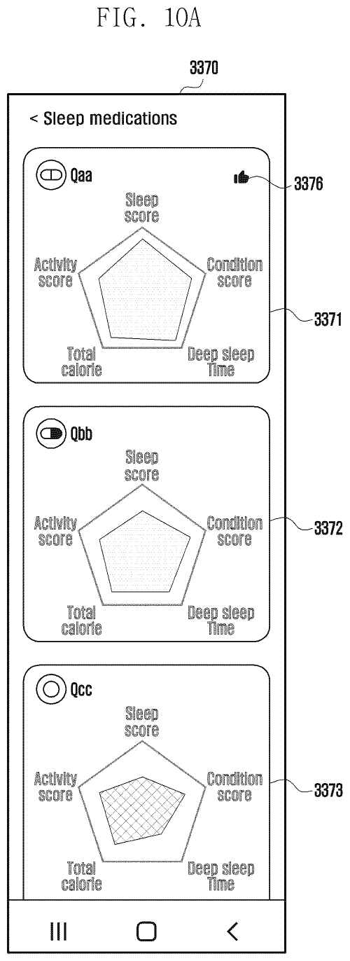

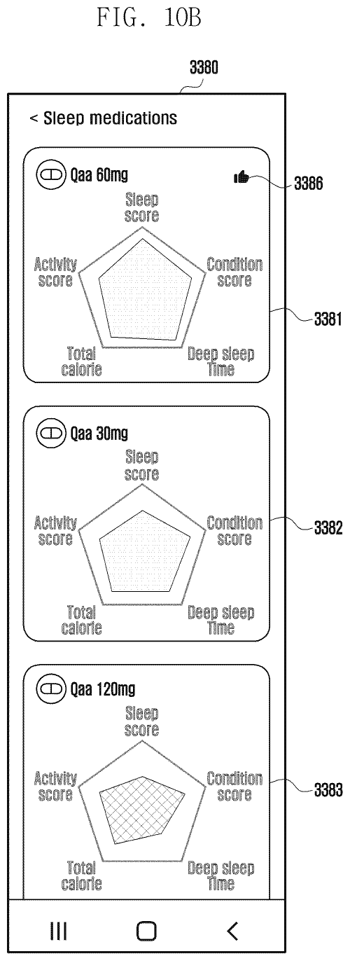

FIGS. 10A and 10B are diagrams illustrating an example operation of providing information on related medications according to various embodiments.

With reference to FIG. 10A, a UI 3370 providing information on medications related to a taken medication is illustrated. For example, the electronic device 200 may store a user's medication dosage history and analysis data of the taken medication. The electronic device 200 may receive a user's input of requesting related information. Based on the stored medication dosage history, the electronic device 200 may determine analysis data of other medications included in the same medication category as the taken medication. For example, if the taken medication is a sleeping pill, the same medication category may be a sleeping pill category. The electronic device 200 may generate and display a visual object according to the analysis data of other medications included in the same medication category, based on the effects and/or side effects. In an example, the electronic device 200 may determine the effects of the other medications, based on the area of the visual object of the analysis data of the other medication included in the same medication category. In an example, the electronic device 200 may determine the effect of the other medication, based on at least one of the first score according to the first factor and/or the second score according to the second factor of the analysis data. In an example, the electronic device 200 may determine the effect of the other medication, based on the weight assigned for the first factor and/or the second factor of the analysis data. The electronic device 200 may provide visual objects on a given number of medications having the best effects. In an example, the electronic device 200 may provide visual objects on a given number of medications having the least number and/or frequency of side effects.

For example, the electronic device 200 may provide visual objects of other medications, including the visual object of the medication being taken. In an example, if the sleeping pill being taken is Qaa, and if Qbb and Qcc have better effects or have fewer side effects among sleeping pills included in the same medication category, the electronic device 200 may provide a visual object 3371 based on the analysis data of Qaa, a visual object 3372 based on the analysis data of Qbb, and a visual object 3373 based on the analysis data of Qcc. For example, the electronic device 200 may provide visual objects of other medications, excluding the visual object of the medication being taken. In an example, if the sleeping pill being taken is QQQ, and if Qaa, Qbb, and Qcc have better effects or have fewer side effects among the sleeping pills included in the same medication category, the electronic device 200 may provide the visual object 3371 based on the analysis data of Qaa, the visual object 3372 based on the analysis data of Qbb, and the visual object 3373 based on the analysis data of Qcc.

The electronic device 200 may display an indicator 3376 recommending the medication with the best effects or the least side effects.

For example, the electronic device 200 may provide information on medications taken by other persons. In an example, the electronic device 200 may provide information on other medications, based on a dosage history and analysis data of medications of the registered family. The electronic device 200 may connect to a family's terminal device and/or an external device and check the dosage history and analysis data of medications of the family. The medication analysis data checked by the electronic device 200 may be analysis data of other medications included in the same medication category as the medication taken by the user. Based on the dosage history and analysis data of medications of the family, the electronic device 200 may generate and display a visual object based on effects and/or side effects. The electronic device 200 may provide visual objects on a given number of medications having the best effects. In an example, the electronic device 200 may provide visual objects on a given number of medications having the least number and/or frequency of side effects. The electronic device 200 may provide the visual object(s) of at least one other medication on the UI 3370 that provides information on medications. The electronic device 200 may display the indicator 3376 recommending the medication with the best effects or the least side effects. In an example, the electronic device 200 may provide the visual object of the medication being taken and the visual object of other medication together on the UI 3370 that provides information on medications.

In an example, the electronic device 200 may provide information on other medications, based on the medication dosage history and medication analysis data of other person with a predetermined correlation or higher. In an example, the predetermined correlation may include a correlation of overall biometric information between the user and the other person, a correlation of biometric information related to symptoms between the user and the other person, a correlation of first and second factors between the user and the other person, and/or a correlation of side effects between the user and the other person. The electronic device 200 may connect to an external device and check the medication dosage history and medication analysis data of other person with a predetermined correlation or higher. The medication analysis data checked by the electronic device 200 may be analysis data of other medication included in the same medication category as the medication being taken by the user. Based on the medication dosage history and medication analysis data of other person with a predetermined correlation or higher, the electronic device 200 may generate and display visual objects based on effects and/or side effects. The electronic device 200 may provide visual objects on a given number of medications having the best effects. In an example, the electronic device 200 may provide visual objects on a given number of medications having the least number and/or frequency of side effects. The electronic device 200 may display the visual object(s) of at least one other medication on the UI 3370 that provides information on medications. The electronic device 200 may display the indicator 3376 recommending the medication with the best effects or the least side effects.

For example, using a learned artificial intelligence model, the electronic device 200 may provide information on a medication included in a medication category according to a symptom entered by the user based on effects and/or side effects. In an example, the artificial intelligence model may learn the medication dosage histories of the user, family, and/or an unspecified number of other persons. In an example, the artificial intelligence model may learn the analysis data of the user, family, and/or an unspecified number of other persons. The medication information provided by the learned artificial intelligence model may be information on a single medication or information on a combination of multiple medications. In an example, if the combination of multiple medications includes a main medication and a sub medication, the electronic device 200 may discriminatively display the main medication and the sub medication. In an example, the electronic device 200 may discriminate the main medication and the sub medication using different colors, line thicknesses, and/or sizes. In an example, the electronic device 200 may display the main medication in a large size or with a thick line.

With reference to FIG. 10B, a UI 3380 providing analysis data of different dosages is illustrated. The electronic device 200 may provide a plurality of visual objects of the same medication with different dosages. In an example, the electronic device 200 may provide a first visual object 3381 when Qaa is taken at 60 mg, a second visual object 3382 when Qaa is taken at 30 mg, and a third visual object 3383 when Qaa is taken at 120 mg. The electronic device 200 may display an indicator 3386 that recommends a medication with the best effect or the least side effect.

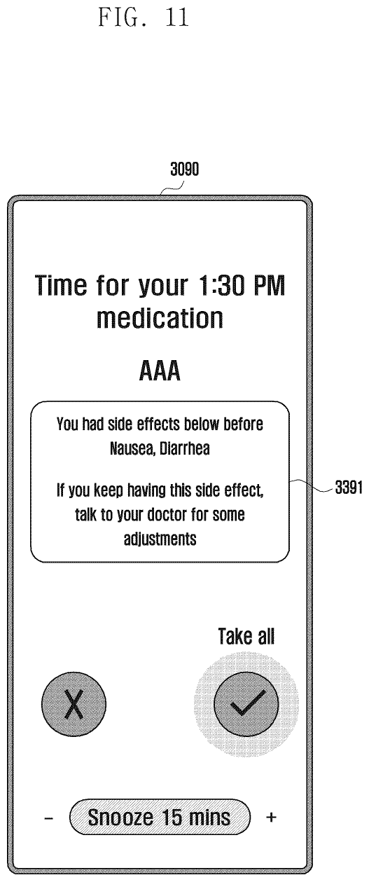

FIG. 11 is a diagram illustrating an example dosage time reminder UI after receiving side effect information according to various embodiments.

With reference to FIG. 11, a dosage reminder UI 3090 is illustrated. The electronic device 200 may provide a medication dosage reminder to the user, based on an input medication dosage schedule. For example, if the medication dosage schedule for AAA medication is 1:30 PM, the electronic device 200 may provide a medication dosage reminder for AAA medication at 1:30 PM. The electronic device 200 may display the dosage reminder UI 3090 and output a reminder sound and/or vibration.

After receiving side effect information, the electronic device 200 may provide a notification 3391 related to the side effect on the dosage reminder UI 3090. In an example, the electronic device 200 may provide a notification suggesting an inquiry to a doctor or pharmacist along with the received side effect information.

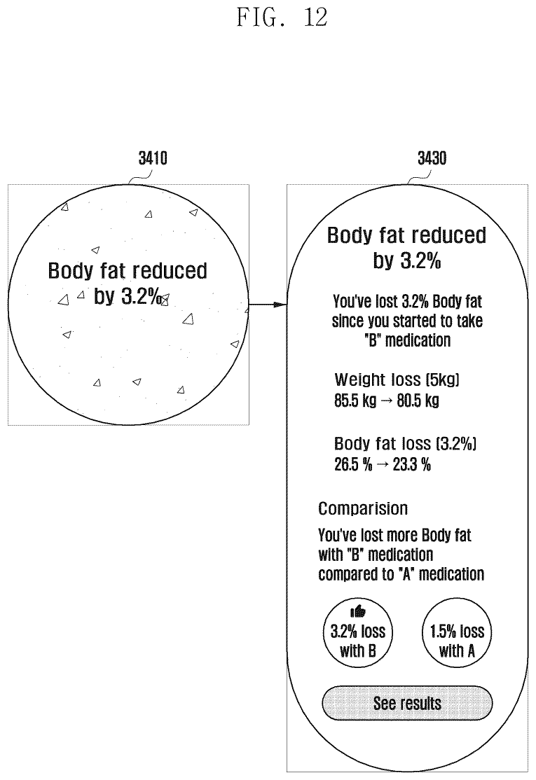

FIG. 12 is a diagram illustrating an example operation of providing comparative data before and after taking a medication according to various embodiments.

With reference to FIG. 12, comparative data before and after taking a medication is illustrated. For example, the electronic device 200 may provide a first type UI 3410. In an example, the first type UI 3410 may be a UI that contains brief information. Upon receiving a user input in the first type UI 3410, the electronic device 200 may provide a second type UI 3430. In an example, the second type UI 3430 may be a UI that contains detailed information.

For example, if a medication is related to weight loss, the electronic device 200 may display comparative data including weight information before and after taking the medication. If a medication is related to insomnia, the electronic device 200 may display comparative data including deep sleep time before and after taking the medication. If a medication is related to blood pressure, the electronic device 200 may display comparative data including blood pressure information before and after taking the medication. If a medication is related to diabetes, the electronic device 200 may display comparative data including blood sugar information before and after taking the medication. If a medication is related to arrhythmia, the electronic device 200 may display comparative data including the degree of pulse regularity before and after taking the medication.

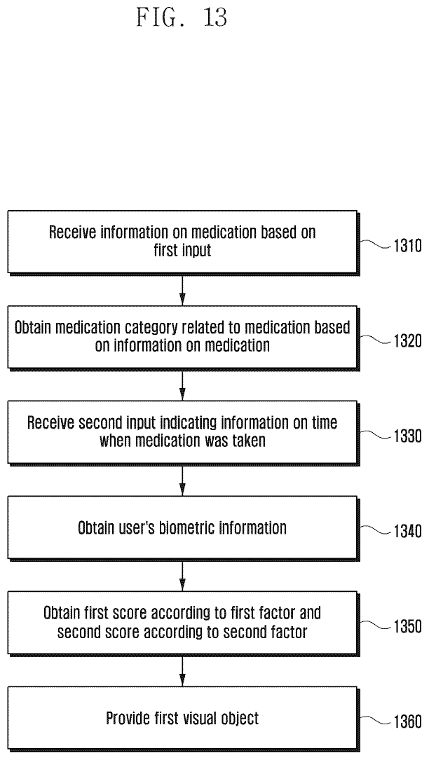

FIG. 13 is a flowchart illustrating an example method for providing information on a taken medication according to various embodiments.

In the following example, respective operations may be performed sequentially, but is not necessarily performed sequentially. For example, the order of operations may be changed, and at least two operations may be performed in parallel.

According to an embodiment, operations 1310 to 1360 may be understood to be performed by a processor (e.g., the processor 230 in FIG. 2) of an electronic device (e.g., the electronic device 200 in FIG. 2).

With reference to FIG. 13, in 1310, the electronic device 200 may receive information on a medication based on a first input (e.g., first user input). In an example, the electronic device 200 may receive the name of a medication directly from the user and receive information on the medication based on the received medication name. If an input using a camera is received, the electronic device 200 may identify the medication name using the camera and receive information on the medication based on the identified medication name. The electronic device 200 may receive a prescription and receive information on the medication based on the received prescription.

In 1320, the electronic device 200 may obtain a medication category related to the medication based on the information on the medication. For example, the electronic device 200 may obtain classification information on the medication from a database connected through an external device (e.g., a server) or obtain the medication category based on the information on the medication. For example, the medication category may include a first medication category and a second medication category. The first medication category may be a medication category that contains analysis data including qualitative information (e.g., self-diagnosis information), and the second medication category may be a medication category that contains analysis data including quantitative information. In an example, if the medication taken is a medication related to pain relief, the analysis data may include qualitative information including the degree of pain symptom relief, and thus the medication related to pain relief may be classified as the first medication category. If the medication taken is a medication related to weight loss, the analysis data may include weight loss information, and thus the medication related to weight loss may be classified as the second medication category. The medications classified as the first medication category or the second medication category may be classified into detailed medication categories according to the type of the medication and/or the corresponding symptom. For example, if the medication taken is a headache medication, it may be classified as the first medication category and the headache medication or pain reliever category.

In 1330, the electronic device 200 may receive a second input (e.g., second user input) indicating information on a time when the medication was taken. In an example, the information on the medication may include a dosage schedule. The electronic device 200 may provide a dosage reminder to the user, based on the dosage schedule. In an example, a dosage reminder UI may contain an item for entering whether the medication is taken. The electronic device 200 may receive a dosage completion input (e.g., a second user input) from the user. For example, the electronic device 200 may receive the dosage completion input containing a time when the medication was taken, entered directly by the user. For example, the electronic device 200 may regard, as time information when the medication was taken, current time information of the electronic device 200 when receiving the dosage completion input from the user. The dosage completion input received from the user may indicate the time information when the medication was taken.

In 1340, the electronic device 200 may obtain user's biometric information. For example, the electronic device 200 may continuously obtain the biometric information periodically or at a predetermined time. In an example, the predetermined time may include a dosage time of the medication or an effect onset time of the medication.

In 1350, the electronic device 200 may obtain a first score according to a first factor and a second score according to a second factor. For example, the first factor may include a factor commonly applicable to a plurality of medication categories. In an example, the first factor may include activity information, sleep information, condition information, and/or calorie information. The first score according to the first factor may include a score that quantifies the first factor. For example, the second factor may include a factor applicable to a medication category related to medication. In an example, the second factor may include weight information, blood sugar information, blood pressure information, heart rate information, sleep information, and/or self-diagnosis information. The second score according to the second factor may include a score that quantifies the second factor. The electronic device 200 may obtain the first score according to the first factor and the second score according to the second factor, based on the obtained biometric information.

For example, if the medication is classified as the first medication category, the electronic device 200 may determine the effect onset time from the dosage time of the medication. The electronic device 200 may receive the self-diagnosis information from the user after the determined effect onset time. For example, if the medication is a medication related to pain relief, the electronic device 200 may receive the self-diagnosis information including the degree of pain symptom relief, and provide the received self-diagnosis information as the second factor.

For example, if the medication is related to weight loss, the electronic device 200 may determine reduced weight based on the user's biometric information. The electronic device 200 may provide weight information including the reduced weight as the second factor. For example, if the medication is related to insomnia, the electronic device 200 may determine a deep sleep time based on the user's biometric information. The electronic device 200 may provide sleep information including the deep sleep time as the second factor.

In 1360, the electronic device 200 may provide a first visual object. The electronic device 200 may provide the visual object that represents analysis data including the first score according to the first factor and the second score according to the second factor. For example, the visual object may include a chart, a tree map, a heat map, an analysis table, a datasheet, a graph, and/or a histogram.

For example, if the medication is classified as the first medication category, the electronic device 200 may provide the visual object including self-diagnosis information as the second factor. If the medication is a medication related to weight loss, the electronic device 200 may provide the visual object including weight information including reduced weight as the second factor. If the medication is a medication related to insomnia, the electronic device 200 may provide the visual object including sleep information including a deep sleep time as the second factor.

For example, the electronic device 200 may provide information on other medications related to the taken medication and/or symptom. In an example, based on the user's medication dosage history, the electronic device 200 may determine analysis data of other medications included in the same medication category as the taken medication. The electronic device 200 may generate and provide a visual object based on effects and/or side effects from the analysis data of other medications. In an example, the electronic device 200 may provide, as the visual object, analysis data of a family member who has taken the medication and/or analysis data of other person with a predetermined correlation or higher. In an example, using analysis data of medications learned by an artificial intelligence model, the electronic device 200 may provide information on the medication included in the medication category according to a symptom entered by the user based on effects and/or side effects.

In an example, an electronic device may include a display, at least one processor including a processing circuitry, and memory storing instructions, wherein the instructions, when executed by the at least one processor individually or collectively, cause the electronic device to: receive information on a medication, based on a first input; obtain a medication category related to the medication, based on the information on the medication; receive a second input indicating information on a time at which the medication was taken; obtain biometric information of a user; based on the biometric information and the information on the time at which the medication was taken, obtain a first score according to a first factor applicable to a plurality of medication categories and a second score according to a second factor applicable to the obtained medication category related to the medication; and provide, through the display, a first visual object indicating the first score according to the first factor and the second score according to the second factor.

In an example, the first factor may include at least one of activity information, sleep information, condition information, and calorie information. The second factor may include at least one of weight information, blood sugar information, blood pressure information, heart rate information, sleep information, and self-diagnosis information.

In an example, the instructions, when executed by the at least one processor individually or collectively, cause the electronic device to: determine, based on the medication being classified as a first category, an effect onset time from the time at which the medication was taken, to receive, based on the effect onset time being satisfied, the self-diagnosis information from the user, and to provide the first visual object including the self-diagnosis information as the second factor.

In an example, the instructions, when executed by the at least one processor individually or collectively, cause the electronic device to: receive, based on the medication category being related to pain relief, the self-diagnosis information including a degree of pain symptom relief, and provide the self-diagnosis information as the second score according to the second factor.

In an example, the instructions, when executed by the at least one processor individually or collectively, cause the electronic device to: determine, based on the medication category being related to weight loss, a reduced weight based on the biometric information, and provide the weight information including the reduced weight as the second score according to the second factor.

In an example, the instructions, when executed by the at least one processor individually or collectively, cause the electronic device to: determine, based on the medication category being related to insomnia, a deep sleep time based on the biometric information, and provide the sleep information including the deep sleep time as the second factor.

In an example, the memory may store a dosage history of a medication previously taken by the user and analysis data on the previously taken medication. The instructions, when executed by the at least one processor individually or collectively, cause the electronic device to: receive an input indicating information on a time at which other medication was taken, determine analysis data of the other medication included in the medication category containing the medication based on the dosage history of the medication in response to additional inputs, generate a second visual object based on at least one of effect and side effect from the analysis data of the other medication, and provide the second visual object through the display.

In an example, the instructions, when executed by the at least one processor individually or collectively, cause the electronic device to: provide, as a third visual object, at least one of analysis data of a family member taking the medication and analysis data of another person with a specified correlation or higher.

In an example, the instructions, when executed by the at least one processor individually or collectively, cause the electronic device to: provide information on a medication included in a medication category according to symptom entered by the user based on at least one of effect and side effect using analysis data of medications learned by an artificial intelligence model.

In an example, the instructions, when executed by the at least one processor individually or collectively, cause the electronic device to: receive information on side effects of the medication, store biometric information related to the side effects, obtain the biometric information of the user periodically, and provide, based on the obtained biometric information of the user being within a specified range from the biometric information related to the side effects, a notification.

In an example, the instructions, when executed by the at least one processor individually or collectively, cause the electronic device to: obtain the most recently updated first and second scores based on the biometric information obtained after an effect onset time of the taken medication or a dosage time of the taken medication.

In an example, the electronic device may include a smartphone. The instructions, when executed by the at least one processor individually or collectively, cause the electronic device to obtain the biometric information from an external device.

In an example, the electronic device may include a wearable device. The instructions, when executed by the at least one processor individually or collectively, cause the electronic device to: provide, based on data comprising at least one of the first visual object, information related to the first factor, and information related to the second factor being more than a specified number, the data to an external device.

In an example, a method for providing information on a medication may include: receiving information on a medication, based on a first input; obtaining a medication category related to the medication, based on the information on the medication; receiving a second input indicating information on a time at which the medication was taken; obtaining biometric information of a user; based on the biometric information and the information on the time at which the medication was taken, obtaining a first score according to a first factor applicable to a plurality of medication categories and a second score according to a second factor applicable to the obtained medication category related to the medication; and providing, through the display, a first visual object indicating the first score according to the first factor and the second score according to the second factor.

In an example, the first factor may include at least one of activity information, sleep information, condition information, and calorie information. The second factor may include at least one of weight information, blood sugar information, blood pressure information, heart rate information, sleep information, and self-diagnosis information.

In an example, the method may further include: determining, based on the medication being classified as a first category, an effect onset time from the time when the medication was taken; receiving, based on the effect onset time being satisfied, the self-diagnosis information from the user; wherein providing the first visual object may include providing the first visual object including the self-diagnosis information as the second factor.

In an example, receiving the self-diagnosis information may include: receiving, based on the medication category being related to pain relief, the self-diagnosis information including a degree of pain symptom relief; and providing the first visual object may include providing the self-diagnosis information as the second score according to the second factor.

In an example, the method may further include: determining, in based on the medication category being related to weight loss, a reduced weight based on the biometric information; and providing the first visual object may include providing the weight information including the reduced weight as the second score according to the second factor.

In an example, the method may further include: determining, based on the medication category being related to insomnia, a deep sleep time based on the biometric information; and providing the first visual object may include providing the sleep information including the deep sleep time as the second factor.

In an example, a non-transitory computer-readable storage medium having a program recorded thereon, wherein the program, when executed by at least one processor, comprising processing circuitry, of an electronic device, individually and/or collectively, causes the electronic device to perform a method for providing information on a medication, the method may include: receiving information on a medication, based on a first input; obtaining a medication category related to the medication, based on the information on the medication; receiving a second input indicating information on a time at which the medication was taken; obtaining biometric information of a user; based on the biometric information and the information on the time at which the medication was taken, obtaining a first score according to a first factor applicable to a plurality of medication categories and a second score according to a second factor applicable to the obtained medication category related to the medication; and providing, through the display, a first visual object indicating the first score according to the first factor and the second score according to the second factor.

It should be appreciated that various examples of the disclosure and the terms used therein are not intended to limit the technological features set forth herein to particular examples and include various changes, equivalents, or replacements for a corresponding example. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “Ist” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it denotes that the element may be coupled with the other element directly (e.g., wiredly), wirelessly, or via a third element.

As used in connection with various examples of the disclosure, the term “module” may include a unit implemented in hardware, software, or firmware, or any combination thereof, and may interchangeably be used with other terms, for example, “logic,” “logic block,” “part,” or “circuitry”. A module may be a single integral component, or a minimum unit or part thereof, adapted to perform one or more functions. For example, according to an example, the module may be implemented in a form of an application-specific integrated circuit (ASIC).

Various examples as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a compiler or a code executable by an interpreter. The machine-readable storage medium may be provided in the form of a non-transitory storage medium. Wherein, the “non-transitory” storage medium is a tangible device, and may not include a signal (e.g., an electromagnetic wave), but this term does not differentiate between where data is semi-permanently stored in the storage medium and where the data is temporarily stored in the storage medium.

According to an example, a method according to various examples of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various examples, each component (e.g., a module or a program) of the above-described components may include a single entity or multiple entities, and some of the multiple entities may be separately disposed in different components. According to various examples, one or more of the above-described components may be omitted, or one or more other components may be added. Alternatively or additionally, a plurality of components (e.g., modules or programs) may be integrated into a single component. In such a case, according to various examples, the integrated component may still perform one or more functions of each of the plurality of components in the same or similar manner as they are performed by a corresponding one of the plurality of components before the integration. According to various examples, operations performed by the module, the program, or another component may be carried out sequentially, in parallel, repeatedly, or heuristically, or one or more of the operations may be executed in a different order or omitted, or one or more other operations may be added.

The effects of the disclosure are not limited to those mentioned above, and other effects not mentioned will be clearly understood by those skilled in the art from the above description. While the disclosure has been illustrated and described with reference to various example embodiments, it will be understood that the various example embodiments are intended to be illustrative, not limiting. It will be further understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein.

What is claimed is:

1. An electronic device comprising:
a display;
at least one processor comprising processing circuitry; and
memory storing instructions,
wherein the instructions, when executed by the at least one processor individually or collectively, cause the electronic device to:
receive information on a medication, based on a first input,
obtain a medication category related to the medication, based on the information on the medication,
receive a second input indicating information on a time at which the medication was taken,
obtain biometric information of a user,
based on the biometric information and the information on the time at which the medication was taken, obtain a first score according to a first factor applicable to a plurality of medication categories and a second score according to a second factor applicable to the obtained medication category related to the medication, and
provide, through the display, a first visual object indicating the first score according to the first factor and the second score according to the second factor.

2. The electronic device of claim 1, wherein the first factor comprises at least one of activity information, sleep information, condition information, and calorie information, and
wherein the second factor comprises at least one of weight information, blood sugar information, blood pressure information, heart rate information, sleep information, and self-diagnosis information.

3. The electronic device of claim 2, wherein the instructions, when executed by the at least one processor individually or collectively, cause the electronic device to:
determine, based on the medication being classified as a first category, an effect onset time from the time at which the medication was taken,
receive, based on the effect onset time being satisfied, the self-diagnosis information from the user, and
provide the first visual object including the self-diagnosis information as the second factor.

4. The electronic device of claim 3, wherein the instructions, when executed by the at least one processor individually or collectively, cause the electronic device to:
receive, based on the medication category being related to pain relief, the self-diagnosis information including a degree of pain symptom relief, and
provide the self-diagnosis information as the second score according to the second factor.

5. The electronic device of claim 2, wherein the instructions, when executed by the at least one processor individually or collectively, cause the electronic device to:
determine, based on the medication category being related to weight loss, a reduced weight based on the biometric information, and
provide the weight information including the reduced weight as the second score according to the second factor.

6. The electronic device of claim 2, wherein the instructions, when executed by the at least one processor individually or collectively, cause the electronic device to:
determine, based on the medication category being related to insomnia, a deep sleep time based on the biometric information, and
provide the sleep information including the deep sleep time as the second factor.

7. The electronic device of claim 1, wherein the memory stores a dosage history of a medication previously taken by the user and analysis data on the previously taken medication, and
wherein the instructions, when executed by the at least one processor individually or collectively, cause the electronic device to:
receive an input indicating information on a time at which other medication was taken,
determine analysis data of the other medication included in the medication category containing the medication based on the dosage history of the medication in response to additional inputs,
generate a second visual object based on at least one of effect and side effect from the analysis data of the other medication, and
provide the second visual object through the display.

8. The electronic device of claim 1, wherein the instructions, when executed by the at least one processor individually or collectively, cause the electronic device to:
provide, as a third visual object, at least one of analysis data of a family member taking the medication and analysis data of another person with a specified correlation or higher.

9. The electronic device of claim 1, wherein the instructions, when executed by the at least one processor individually or collectively, cause the electronic device to:
provide information on a medication included in a medication category according to symptom entered by the user based on at least one of effect and side effect using analysis data of medications learned by an artificial intelligence model.

10. The electronic device of claim 1, wherein the instructions, when executed by the at least one processor individually or collectively, cause the electronic device to:
receive information on side effects of the medication,
store biometric information related to the side effects,
obtain the biometric information of the user periodically, and
provide, based on the obtained biometric information of the user being within a specified range from the biometric information related to the side effects, a notification.

11. The electronic device of claim 1, wherein the instructions, when executed by the at least one processor individually or collectively, cause the electronic device to:
obtain the most recently updated first and second scores based on the biometric information obtained after an effect onset time of the taken medication or a dosage time of the taken medication.

12. The electronic device of claim 1, wherein the electronic device comprises a smartphone, and
wherein the instructions, when executed by the at least one processor individually or collectively, cause the electronic device to:
obtain the biometric information from an external device.

13. The electronic device of claim 1, wherein the electronic device comprises a wearable device, and
wherein the instructions, when executed by the at least one processor individually or collectively, cause the electronic device to:
provide, based on data comprising at least one of the first visual object, information related to the first factor, and information related to the second factor being more than a specified number, the data to an external device.

14. A method for providing information on a medication, the method comprising:
receiving information on a medication, based on a first input;
obtaining a medication category related to the medication, based on the information on the medication;
receiving a second input indicating information on a time at which the medication was taken;
obtaining biometric information of a user;
based on the biometric information and the information on the time at which the medication was taken, obtaining a first score according to a first factor applicable to a plurality of medication categories and a second score according to a second factor applicable to the obtained medication category related to the medication; and
providing, through the display, a first visual object indicating the first score according to the first factor and the second score according to the second factor.

15. The method of claim 14, wherein the first factor comprises at least one of activity information, sleep information, condition information, and calorie information, and
wherein the second factor comprises at least one of weight information, blood sugar information, blood pressure information, heart rate information, sleep information, and self-diagnosis information.
